(19) United States
(12) Patent Application Publication
(10) Pub. No.: US 2026/0174888 A1
(43) Pub. Date: Jun. 25, 2026

(54) DUAL PAYLOAD ANTIBODY DRUG CONJUGATES FOR THE TREATMENT

(71) Applicant: ARARIS BIOTECH AG, Au (CH)

(72) Inventors: Isabella ATTINGER-TOLLER, Au (CH); Romain BERTRAND, Au (CH); Rachael FAY, Au (CH); Philipp PROBST, Au (CH); Roger SANTIMARIA, Au (CH); Dragan GRABULOVSKI, Au (CH); Philipp SPYCHER, Au (CH)

(21) Appl. No.: 19/389,744

(22) Filed: Nov. 14, 2025

Related U.S. Application Data
(63) Continuation of application No. PCT/EP2024/065351, filed on Jun. 4, 2024.

(30) Foreign Application Priority Data
Jun. 4, 2023 (EP) 23177132.0

(51) Int. Cl. A61K 47/68; A61K 47/55; A61P 35/00

(52) U.S. Cl. CPC A61K 47/68031; A61K 47/551; A61K 47/6853; A61K 47/6889; A61P 35/00

(57) ABSTRACT

The present invention relates to an antibody-drug conjugate (ADC) comprising as a first payload a cytotoxic molecule and as a second payload a tumor-targeting small molecule for use in the treatment of cancer, wherein the cancer is characterized by the presence of a cancer cell comprising a first antigen that is specifically bound by an antibody portion of the ADC and a second antigen that specifically interacts with the tumor-targeting small molecule.

### RELATED APPLICATIONS

This application is a continuation of International Patent Application No. PCT/EP2024/065351, filed Jun. 4, 2024, which claims priority to European Patent Application No. 23177132.0, filed Jun. 4, 2023, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Nov. 13, 2025, is named 770379_ARA9-009PCCON_ST26.xml and is 5,802 bytes in size.

The present invention relates to the field of cancer immunotherapy and is concerned with the use of antibody-drug conjugates (ADCs) comprising at least two payloads, wherein the first payload is a cytotoxic molecule, and the second payload is a tumor-targeting small molecule.

ADCs are a type of targeted cancer therapy that combines the specificity of monoclonal antibodies with the cytotoxic potency of chemotherapeutic drugs. ADCs consist of three main components: a monoclonal antibody that targets a specific antigen on cancer cells, a cytotoxic drug that kills the cancer cells, and a linker that connects the antibody and the drug.

The monoclonal antibody recognizes and binds to a specific antigen on the surface of cancer cells, allowing the ADC to specifically target cancer cells while sparing healthy cells. Once the ADC binds to the cancer cell, the linker is cleaved, releasing the cytotoxic drug into the cancer cell, which then kills the cancer cell.

ADCs are designed to maximize the therapeutic effect on cancer cells while minimizing the toxicity to healthy cells, resulting in fewer side effects compared to traditional chemotherapy. They are an emerging class of targeted therapies that have shown promising results in the treatment of various types of cancer. However, while antibody-drug conjugates (ADCs) have shown great promise as a targeted cancer therapy, there are several challenges associated with their development and use. Some of the main challenges of ADCs are:
1. Design and optimization: The design and optimization of ADCs is a complex process that involves selecting the appropriate antibody, linker, and cytotoxic drug, and optimizing the ratios between these components to achieve optimal therapeutic efficacy. This process requires a deep understanding of the pharmacokinetics and pharmacodynamics of each component, as well as their interactions with each other.
2. Heterogeneity: ADCs can be heterogeneous, meaning that different ADC molecules can have different numbers of drugs attached to the antibody or different types of linkers. This can lead to variability in drug potency and pharmacokinetics, making it difficult to establish optimal dosing regimens.
3. Stability: ADCs can be prone to instability, which can lead to premature release of the drug or degradation of the antibody. This can affect the efficacy and safety of the therapy, and can also impact the shelf life of the product.
4. Immunogenicity: ADCs can be immunogenic, meaning that they can stimulate an immune response in the patient. This can lead to the production of anti-drug antibodies (ADAs), which can reduce the efficacy of the therapy and increase the risk of adverse reactions.
5. Manufacturing: The manufacturing of ADCs is a complex process that requires specialized equipment and expertise. The process involves multiple steps, including antibody production, drug synthesis, linker conjugation, and purification, and requires strict quality control measures to ensure product consistency and safety.

Internalization into tumor cells is another key aspect in the mechanism of action of antibody-drug conjugates (ADCs). The monoclonal antibody component of the ADC specifically targets and binds to a surface antigen on the tumor cell, allowing the entire ADC molecule to be internalized by the cell through receptor-mediated endocytosis. Once inside the tumor cell, the ADC is trafficked to lysosomes, which are organelles that contain enzymes that break down macromolecules. The acidic environment of the lysosome triggers the cleavage of the linker that connects the antibody and the cytotoxic drug, releasing the drug into the cytoplasm of the tumor cell. The cytotoxic drug then exerts its pharmacological effect, typically by disrupting critical cellular processes such as DNA replication or microtubule assembly, leading to tumor cell death. Therefore, the internalization of the ADC into tumor cells is critical for the drug to be released and exert its pharmacological effect. The specificity of the monoclonal antibody for the tumor antigen ensures that the cytotoxic drug is delivered preferentially to tumor cells, minimizing toxicity to healthy cells.

However, there are several challenges associated with the internalization of antibody-drug conjugates (ADCs) into tumor cells: 1. Antigen expression: The level of antigen expression on the surface of tumor cells can vary widely, and not all tumor cells may express the antigen targeted by the ADC. This can limit the effectiveness of the therapy and reduce the internalization of the ADC into tumor cells.
2. Antigen accessibility: Even if the antigen is expressed on the surface of tumor cells, it may be located in regions that are not easily accessible to the monoclonal antibody component of the ADC. This can limit the binding and internalization of the ADC into tumor cells.
3. Resistance: Some tumor cells may develop resistance to ADCs by reducing or altering the expression of the targeted antigen, or by developing mechanisms to prevent internalization of the ADC.

There are several strategies that can be used to enhance the internalization of antibody-drug conjugates (ADCs) into tumor cells. One approach is to use a cell-penetrating peptide (CPP), which can facilitate the uptake of macromolecules into cells by crossing the plasma membrane. CPPs can be conjugated to the monoclonal antibody component of the ADC to increase its internalization into tumor cells. For example, CPPs have been conjugated to an anti-HER2 antibody-drug conjugate to enhance the internalization of the ADC (Sauter et al, (2020), Journal of Controlled Release, 322, p. 200-208). However, one challenge of this approach is that CPPs can interact with multiple cell surface receptors and undergo non-specific uptake into cells, which can lead to off-target effects and toxicity. Another challenge is that CPPs can be rapidly degraded by proteases and cleared from the body by the kidneys and liver, which can limit their efficacy.

Additionally, other strategies for enhancing internalization of ADCs include the use of bispecific antibodies that can simultaneously target multiple antigens on tumor cells (see for example Zong et al. (2022), Pharmaceut Fronts, 4: e113-e120). However, bispecific ADCs have some unique challenges compared to traditional ADCs, some of which include:
1. Complexity: Bispecific ADCs are more complex than traditional ADCs because they require the incorporation of two different binding domains that can recognize distinct targets. This can lead to challenges in developing and optimizing the manufacturing process.
2. Stability: Bispecific ADCs may have decreased stability compared to traditional ADCs due to the presence of two different binding domains, which can lead to instability or premature degradation.
3. Heterogeneity: Bispecific ADCs can exhibit heterogeneity in terms of the distribution of drug molecules and/or binding domains on the antibody structure, which can affect their potency, pharmacokinetics, and safety profile.
4. Immunogenicity: The presence of two different binding domains in bispecific ADCs can increase their potential to elicit an immune response, which can reduce their effectiveness and/or cause adverse effects.
5. Tissue penetration: Bispecific ADCs may have decreased tissue penetration compared to traditional ADCs due to their larger size and complexity, which can limit their ability to access and target certain tissues or cells.

Therefore, there is a need in the art to identify better solutions to enhance the internalization and delivery of ADCs to cancer cells. The technical problem underlying the present invention can thus be formulated as the provision of ADCs that are efficiently internalized into cancer cells.

### SUMMARY OF THE INVENTION

The present invention is characterized in the herein provided embodiments and claims. In particular, the present invention relates, inter alia, to the following embodiments:

In one embodiment, the invention relates to an antibody-drug conjugate (ADC) comprising as a first payload a cytotoxic molecule and as a second payload a tumor-targeting small molecule for use in the treatment of cancer, wherein the cancer is characterized by the presence of a cancer cell comprising a first antigen that is specifically bound by an antibody portion of the ADC and a second antigen that specifically interacts with the tumor-targeting small molecule.

That is, it has been surprisingly found by the inventors that ADCs comprising as a first payload a cytotoxic molecule and as a second payload a tumor-targeting small molecule, such as a folate molecule, are internalized more efficiently into cancer cells than ADCs that do not comprise the tumor-targeting small molecule (see Example 1 and FIGS. 1A-1B). This enhanced internalization resulted in increased killing activity of the ADC (See Example 2 and FIG. 2).

It was shown by Yamaguchi et al. (Yamaguchi et al. (2021) Bioorg. Med. Chem. 32, 116013) that small molecule-based bispecific antibody-drug conjugates, e.g., a HER2-targeting antibody comprising the cytotoxic molecule MMAF and a small molecule, such as folic acid, as payloads, can broaden the target scope of ADCs. In particular, it was shown that a small molecule-based bispecific antibody was able to kill folate receptor-positive cells that did not express HER2, as well as Her2-positive cells that did not express a folate receptor. Accordingly, the small molecule-based bispecific antibody was proposed by Yamaguchi et al. for the treatment of heterogenous cancers.

In the present invention, it was surprisingly found that ADCs comprising a cytotoxic molecule and a tumor-targeting small molecule as payloads are not only suitable for the treatment of heterogenous cancers, as reported previously be Yamaguchi et al., but are even more effective against double-positive cancer cells that express both an antigen that is specifically bound by the antibody portion of the ADC and an antigen that specifically interacts with the tumor-targeting small molecule.

In further embodiment, the invention relates to the antibody-drug conjugate for use of the invention, wherein the tumor-targeting small molecule facilitates internalization of the antibody-drug conjugate into the cancer cell and/or improves the killing activity of the antibody-drug conjugate against the cancer cell.

In another embodiment, the invention relates to an antibody-drug conjugate (ADC) comprising as a first payload a cytotoxic molecule and as a second payload a tumor-targeting small molecule for use in the treatment of cancer, wherein the tumor-targeting small molecule facilitates internalization of the antibody-drug conjugate into a cancer cell comprising an antigen that specifically interacts with the tumor-targeting small molecule and/or improves the killing activity of the antibody-drug conjugate against a cancer cell comprising an antigen that specifically interacts with the tumor-targeting small molecule.

In further embodiment, the invention relates to the antibody-drug conjugate for use of the invention, wherein the tumor-targeting small molecule is selected from the group consisting of:
Folic acid, or derivatives thereof,
Biotin,
EGF,
Integrin binding proteins,
RGD peptides and their derivatives (iRGD, cilengitide, SFITGv6, CNGRC etc.),
extracellular matrix-homing peptides (DAG, ZD2, CSG, PIGF-2, BT1718),
tumor associated macrophages-targeting agents (RP-182, M2pep, mUNO),
EGFR targeting peptide (GE11),
Angiopep-2,
peptides targeting aberrant cellular signaling pathways (LP4, NBD, H1),
PSMA binders (urea-based or phosphoramidate-based binders), and
FAP binders.

In another embodiment, the invention relates to the antibody-drug conjugate for use of the invention, wherein the tumor-targeting small molecule is folic acid, or a derivative thereof, and wherein the antigen that specifically interacts with the tumor-targeting small molecule is folate receptor alpha (FRα).

In yet another embodiment, the invention relates to the antibody-drug conjugate for use of the invention, wherein the antibody is an IgG antibody, in particular an IgG1 antibody.

In a further embodiment, the invention relates to the antibody-drug conjugate for use of the invention, wherein the ADC has the formula A-L, wherein A is an antibody, or an antibody fragment, and wherein L is a linker comprising the cytotoxic molecule and the tumor-targeting small molecule.

In another embodiment, the invention relates to the antibody-drug conjugate for use of the invention, wherein the linker is conjugated to the side chain of a glutamine residue of the antibody, in particular wherein the linker is conjugated to the side chain of the glutamine residue of the antibody via an isopeptide bond formed between a primary amine comprised in the linker and a carboxyl group comprised in the side chain of the glutamine residue.

In another embodiment, the invention relates to the antibody-drug conjugate for use of the invention, wherein the glutamine residue to which the linker is conjugated is comprised in an Fc domain of the antibody, in particular wherein the glutamine residue to which the linker is conjugated is glutamine residue Q295 (EU numbering) of the CH2 domain of an IgG antibody.

In yet another embodiment, the invention relates to the antibody-drug conjugate for use of the invention, wherein the glutamine residue to which the linker is conjugated has been introduced into the heavy or light chain of the antibody by molecular engineering.

In another embodiment, the invention relates to the antibody-drug conjugate for use of the invention, wherein the glutamine residue that has been introduced into the heavy or light chain of the antibody by molecular engineering is N297Q (EU numbering) of the CH2 domain of an aglycosylated IgG antibody; or
wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

In another embodiment, the invention relates to the antibody-drug conjugate for use of the invention, wherein the linker is conjugated to the side chain of the glutamine residue of the antibody via a lysine residue, a cadaverine moiety or a PEG amine moiety.

In another embodiment, the invention relates to the antibody-drug conjugate for use of the invention, wherein the lysine residue is comprised in a peptide linker.

In yet another embodiment, the invention relates to the antibody-drug conjugate for use of the invention, wherein the peptide linker comprises not more than 25, 20, 15, 14, 13, 12, 11, 10, 9, 8, 7, 6, 5, 4 amino acid residues.

In another embodiment, the invention relates to the antibody-drug conjugate for use of the invention, wherein the net charge of the peptide linker is neutral or positive.

In another embodiment, the invention relates to the antibody-drug conjugate for use of the invention, wherein the peptide linker comprises no negatively-charged amino acid residues.

In another embodiment, the invention relates to the antibody-drug conjugate for use of the invention, wherein the peptide linker comprises at least one of an arginine or histidine residue.

In another embodiment, the invention relates to the antibody-drug conjugate for use of the invention, wherein the peptide linker comprises the sequence motif arginine-lysine (RK) or histidine-lysine (HK).

In another embodiment, the invention relates to the antibody-drug conjugate for use of the invention, wherein the peptide linker comprises the sequence RKAA, RKA, ARK or RK-Val-Cit.

In another embodiment, the invention relates to the antibody-drug conjugate for use of the invention, wherein the tumor-targeting small molecule and/or the cytotoxic molecule are linked to the N- or C-terminus of the peptide linker or to a side-chain of an amino acid residue comprised in the peptide linker.

In yet another embodiment, the invention relates to the antibody-drug conjugate for use of the invention, wherein the tumor-targeting small molecule is linked to the N-terminus of the peptide linker and wherein the cytotoxic molecule is linked to the C-terminus of the peptide linker, or vice versa.

In another embodiment, the invention relates to the antibody-drug conjugate for use of the invention, wherein the tumor-targeting small molecule is linked to the N- or C-terminus of the peptide linker via one or more PEG moieties.

In another embodiment, the invention relates to the antibody-drug conjugate for use of the invention, wherein the peptide linker has the structure:

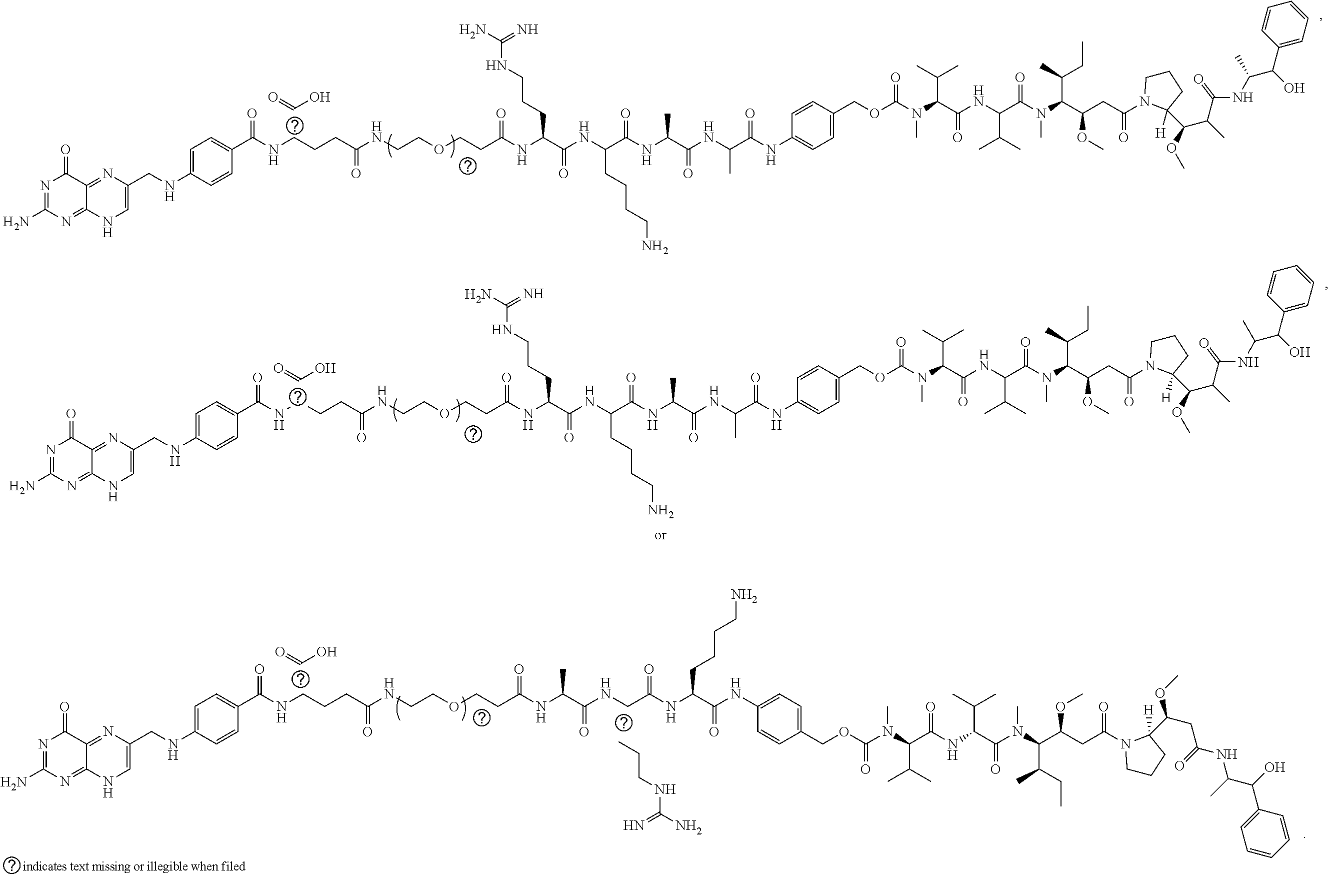

In another embodiment, the invention relates to the antibody-drug conjugate for use of the invention, wherein the linker is conjugated to the side chain of a cysteine residue of the antibody, in particular wherein the linker is conjugated to the side chain of the cysteine residue of the antibody via a thiol-maleimide bond.

In another embodiment, the invention relates to the antibody-drug conjugate for use of the invention, wherein the cytotoxic molecule is linked to the linker or peptide linker via a self-immolative moiety.

In another embodiment, the invention relates to the antibody-drug conjugate for use of the invention, wherein the cytotoxic molecule is at least one selected from the group consisting of
a pyrrolobenzodiazepine (e.g., PBD);
an auristatin (e.g., MMAE, MMAF);
a maytansinoid (e.g., maytansine, DM1, DM4, DM21);
a duocarmycin;
a nicotinamide phosphoribosyltransferase (NAMPT) inhibitor;
a tubulysin;
an enediyne (e.g., calicheamicin);
an anthracycline derivative (PNU) (e.g., doxorubicin);
a pyrrole-based kinesin spindle protein (KSP) inhibitor;
a cryptophycin;
a drug efflux pump inhibitor;
a sandramycin;
an amanitin (e.g., α-amanitin); and
a camptothecin (e.g., exatecans, deruxtecans).

In yet another embodiment, the invention relates to the antibody-drug conjugate for use of the invention, wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4, and/or
wherein the antibody is selected from the group consisting of: Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab and Enfortumab.

In one embodiment, the invention relates to a method of treating cancer in a subject, the method comprising a step of administering an antibody-drug conjugate (ADC) comprising as a first payload a cytotoxic molecule and as a second payload a tumor-targeting small molecule to said subject, wherein the cancer is characterized by the presence of a cancer cell comprising a first antigen that is specifically bound by an antibody portion of the ADC and a second antigen that specifically interacts with the tumor-targeting small molecule.

In another embodiment, the invention relates to a method of treating cancer in a subject, the method comprising a step of administering an antibody-drug conjugate (ADC) comprising as a first payload a cytotoxic molecule and as a second payload a tumor-targeting small molecule to said subject, wherein the tumor-targeting small molecule facilitates internalization of the antibody-drug conjugate into a cancer cell comprising an antigen that specifically interacts with the tumor-targeting small molecule and/or improves the killing activity of the antibody-drug conjugate against a cancer cell comprising an antigen that specifically interacts with the tumor-targeting small molecule.

### BRIEF DESCRIPTION OF THE DRAWINGS

FIGS. 1A-1B: Internalization of ADCs using FA-(PEG) n-RKAA-linkers as of this invention into HTB-133 (Nectin-4+/Fra+)

FIG. 2: Cytotoxicity on NCI-H2110 (CEACAM+/FRα+) cells of ADCs using FA-(PEG) n-RKAA-linkers as of this invention.

FIG. 3: Chemical structure of the linker-payload FA-PEG24-RKAA-PABC-MMAE.

FIG. 4: Chemical structure of the linker-payload FA-PEG12-RKAA-PABC-MMAE.

FIG. 5: Chemical structure of the linker-payload FA-PEG12-ARK-PABC-MMAE.

FIG. 6: Chemical structure of the linker-payload RKAA-PABC-MMAE (not according to this invention)

### EXAMPLES

### Example 1: ADCs Show Superior Internalization Using Internalization-Enhancing Linkers

### Methods

DNA encoding ARA-04, an antibody targeting Nectin-4, was transiently transfected into suspension-adapted CHO-K1 cells and expressed in serum-free/animal component-free media. The proteins were purified from the supernatants by Protein A affinity chromatography (Mab Select Sure column; GE Healthcare). Conjugation reactions were performed by mixing 5 mg/ml of the indicated native, glycosylated antibody, MTG at a concentration of 5-10 U/mg, and 5-10 molar equivalents of the indicated linker-payload, containing folic acid (FA) and optionally a polyethylene glycol (PEG)-spacer, in BisTris 50 mM pH 6.8 for 24 hours at 37° C. in a rotating thermomixer. Control ADC comprising a linker-payload without folic acid (e.g. ADC without internalization-enhancing feature) was prepared using the same conditions. Conjugation efficiency was assessed by LCMS, under DTT reduced conditions. Reduction of samples was achieved by incubation for 15 min at 37° C. in 50 mM DTT (final) and 50 mM Tris buffer. After reduction, samples were analyzed on a Xevo G2-XS QTOF (Waters) coupled to an Acquity UPLC H-Class System (Waters) and an ACQUITY UPLC BEH C18 Column. Conjugation efficiency (CE) was calculated from deconvoluted spectra and presented in %. Intensities resulting from both glycoforms (G1F and GOF) were taken into account for the calculation, according to the formula:

$$CE \% = \frac{\sum ( {( Int ( G 0 F + G 1 F ) )}_{cj} )}{\sum {( Int ( G 0 F + G 1 F ) )}_{cj , ncj}}$$

With cj=conjugated and ncj=non-conjugated

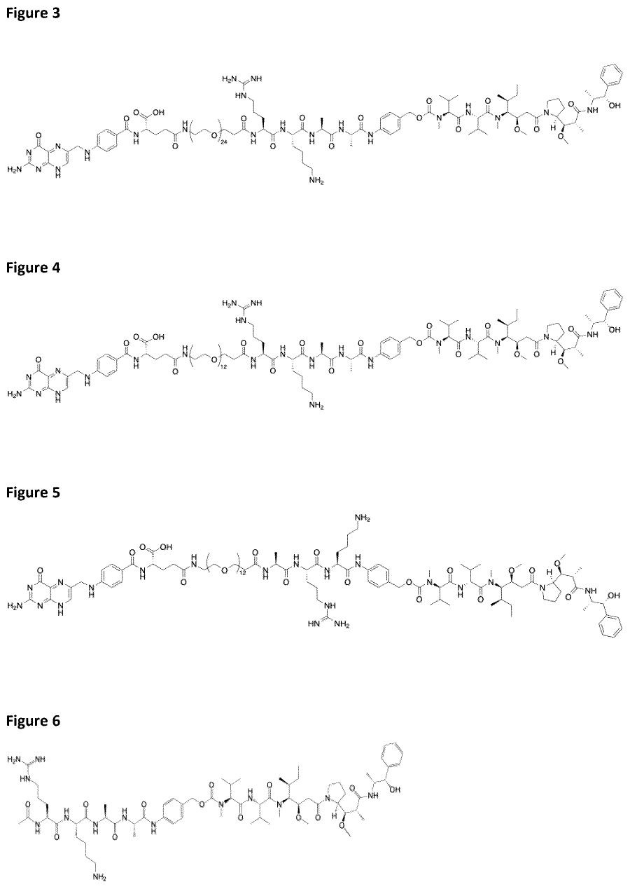

Cell internalization assay was performed using Nectin-4 expressing cells (ATCC, Ref.: HTB-133), stained on ice with ARA-04-folic acid-(polyethylene glycol)₂₄-RKAA-PABC-MMAE (FIG. 3) (=ARA-04-FA-PEG₂₄-RKAA-PABC-MMAE), ARA-04-RKAA-PABC-MMAE, and ARA-04 in a protocol adapted from Barfield, M C B 2020.

Target cell lines (0.5×10⁶ per well) were incubated for 1 hour on ice with the ADCs (1 μg/0.5×10⁶ cells) in BSA/EDTA containing buffer, washed, and placed on ice or at 37° C. for 1 hour to allow for receptor-mediated internalization. Where indicated, a 1000× molar excess of folic acid was added with the ADCs to the cells to saturate binding of Folate Receptor alpha to show contribution of linker-bound FA for internalization.

Then, cells were incubated on ice for 45 minutes with a PE-coupled anti-human IgG Fc antibody, washed twice and measured by flow cytometry on a CytoFlexS FACS instrument (Beckman Coulter). Results were analyzed using FlowJo (v10, Tree Star) The difference in fluorescence between cells at 4 and 37° C. was interpreted as antibody- or ADC-mediated internalization.

### Results

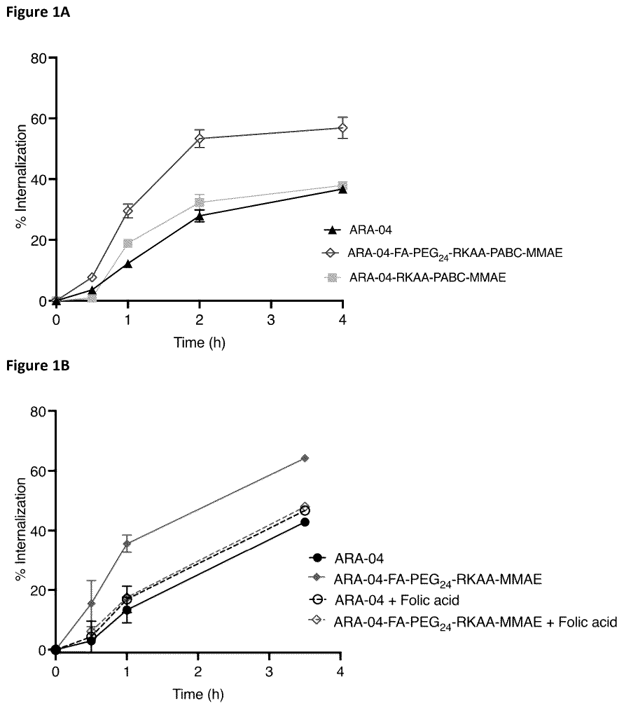

Antibody and ADC internalization is shown as percent in FIGS. 1A and 1B. Internalization of ADC ARA-04-FA-PEG₂₄-RKAA-PABC-MMAE (FIG. 3) comprising internalization-enhancing linkers according to this invention occurs significantly quicker compared to conventional ADC ARA-04-RKAA-PABC-MMAE without internalization-enhancing moiety, or the naked antibody (ARA-04). The surprising increase of approximately 40% higher amount of delivered ADC shows that the use of internalization-enhancing moieties, according to this invention, is significant and a promising approach ADC to optimize efficacy and delivery. Further, FIG. 1B shows that saturation of the folate receptor alpha (FRα) using its ligand, folic acid in 1000× molar excess, prevents increased ADC internalization seen with ARA-04-FA-PEG₂₄-RKAA-PABC-MMAE compared to ARA01-RKAA-PABC-MMAE, demonstrating that the improvement of internalization is indeed mediated by interaction of the FA moiety of the linker described in this application with its receptor, FRα.

### Example 2: Internalization Enhancement Leads to Superior Anti-Tumor Effects In Vitro when Both Targets are Engaged Simultaneously (as of this Invention)

### Methods

DNA encoding ARA-20, an antibody targeting CEACAM-5, was transiently transfected into suspension-adapted CHO-K1 cells and expressed in serum-free/animal component-free media. The proteins were purified from the supernatants by Protein A affinity chromatography (Mab Select Sure column; GE Healthcare). Conjugation reactions were performed using the above-mentioned conditions. Conjugation efficiency was assessed by LCMS as described above.

The growth inhibitory effect of internalization-enhanced ADCs was investigated in vitro on the following Folate-Receptor alpha (FRα) and CEACAM-5 over-expressing cell line: NCI-H2110 (ATCC, Ref. CRL-5924).

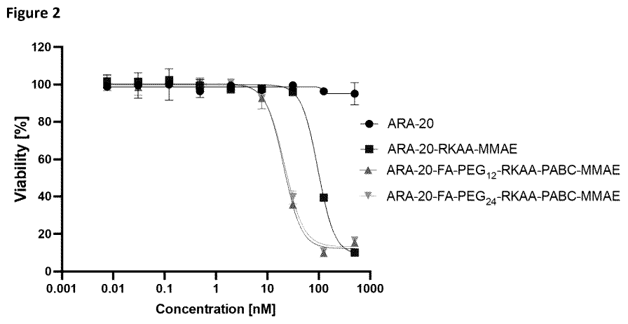

The cytotoxic activity of internalization enhanced ADCs, ARA-20-FA-PEG₁₂-RKAA-PABC-MMAE (FIG. 4), ARA-20-FA-PEG₂₄-RKAA-PABC-MMAE (FIG. 3, FIG. 2A, Table 1) and ARA-20-FA-PEG₁₂-ARK-PABC-MMAE (FIG. 5, FIG. 2B, Table 2) was compared to the conventional ADCs ARA-20-RKAA-PABC-MMAE (FIG. 6, FIG. 2A, Table 1) and ARA-20-ARK-PABC-MMAE (FIG. 2B, Table 2), respectively. For that, 4000 cells were seeded into 96-well culture plates and incubated with ARA-20-FA-PEG₁₂-RKAA-PABC-MMAE, ARA-20-FA-PEG₂₄-RKAA-PABC-MMAE or ARA-20-FA-PEG₁₂-ARK-PABC-MMAE (FIG. 4) and ARA-20-RKAA-PABC-MMAE for 96 hours at 37° C. in a humidified chamber and 5% CO₂.

The viability of the treated cultures was determined by ATP-quantification in a CellTiterGloLuminescence Assay as described by the supplier (Promega). The % viability relative to untreated cells was calculated according to the formula:

$$\% viability = ( \frac{{OD}_{experimental} - {OD}_{blank}}{{OD}_{untreated} - {OD}_{blank}} ) \times 100$$

The average % viability was plotted against log 10 (concentration), and the resulting dose-response curves were analyzed by nonlinear regression with the software Prism8, using a four parameter dose-response curve equation.

### Results

FIG. 2A shows that ARA-20-FA-PEG₁₂-RKAA-PABC-MMAE and ARA-20-FA-PEG₂₄-RKAA-PABC-MMAE had a superior cytotoxic activity against CEACAM-5 over-expressing cells compared to ARA-20-RKAA-PABC-MMAE, without the internalization-enhancing moiety. The same was observed using internalization-enhanced linker ARA-20-FA-PEG₁₂-ARK-PABC-MMAE ADC in comparison to the conventional ADC ARA-20-ARK-PABC-MMAE. The IC50 values of the internalization-optimized ADCs show a 2-5 times more potent cell killing than the respective conventional ADC which is surprising taken the very high potency of MMAE (FIGS. 2A, 2B and Tables 1 and 2). In summary, internalization-enhanced ADCs of this invention, ARA-20-FA-PEG₁₂-RKAA-PABC-MMAE, ARA-20-FA-PEG₂₄-RKAA-PABC-MMAE or ARA-20-FA-PEG₁₂-ARK-PABC-MMAE show significantly enhanced anti-proliferative activity in vitro compared to the non-internalization enhanced counterpart.

TABLE 1
IC50 values of the compounds displayed in FIG. 2A

| mAb or ADC type | IC50 (nM) |
|---|---|
| ARA-20 (mAb only) | NA |
| ARA-20- RKAA-PABC-MMAE (FIG. 6) | 30.2 |
| ARA-20-FA-PEG₁₂-RKAA-PABC-MMAE (FIG. 4) | 4.68 |
| ARA-20-FA-PEG₂₄-RKAA-PABC-MMAE (FIG. 3) | 5.93 |

TABLE 2
IC50 values of the compounds displayed in FIG. 2B

| mAb or ADC type | IC50 (nM) |
|---|---|
| ARA-20 (mAb only) | NA |
| ARA-20-ARK-PABC-MMAE | 58 |
| ARA-20-FA-PEG₁₂-ARK-PABC-MMAE (FIG. 5) | 27.8 |

> ARA-04 Heavy Chain (SEQ ID NO: 1):
EVQLVESGGGLVQPGGSLRLSCAASGFTFSSYNMNWVRQAPGKGLEWVSY
ISSSSSTIYYADSVKGRFTISRDNAKNSLSLQMNSLRDEDTAVYYCARAY
YYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
>ARA-04 Light chain (SEQ ID NO: 2):
DIQMTQSPSSVSASVGDRVTITCRASQGISGWLAWYQQKPGKAPKFLIYA
ASTLNSGVPSRFSGSGSGTDFTLTISSLQPEDFATYYCQQANSFPPTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
> ARA-20 Heavy Chain (SEQ ID NO: 3):
EVQLVESGGGVVQPGRSLRLSCSASGFDFTTYWMSWVRQAPGKGLEWIGE
IHPDSSTINYAPSLKDRFTISRDNAKNTLFLQMDSLRPEDTGVYFCASLY
FGFPWFAYWGQGTPVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
> ARA-20 Light chain (SEQ ID NO: 4):
DIQLTQSPSSLSASVGDRVTITCKASQDVGTSVAWYQQKPGKAPKLLIYW
TSTRHTGVPSRFSGSGSGTDFTFTISSLQPEDIATYYCQQYSLYRSFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

1. A method of treating cancer in a subject in need thereof, comprising administering to said subject an antibody-drug conjugate (ADC) comprising as a first payload a cytotoxic molecule and as a second payload a tumor-targeting small molecule, wherein the cancer is characterized by the presence of a cancer cell comprising a first antigen that is specifically bound by an antibody portion of the ADC and a second antigen that specifically interacts with the tumor-targeting small molecule.

2. The method of claim 1, wherein the tumor-targeting small molecule facilitates internalization of the antibody-drug conjugate into the cancer cell and/or improves the killing activity of the antibody-drug conjugate against the cancer cell.

3. A method of treating cancer in a subject in need thereof, comprising administering to said subject an antibody-drug conjugate (ADC) comprising as a first payload a cytotoxic molecule and as a second payload a tumor-targeting small molecule, wherein the tumor-targeting small molecule facilitates internalization of the antibody-drug conjugate into a cancer cell comprising an antigen that specifically interacts with the tumor-targeting small molecule and/or improves the killing activity of the antibody-drug conjugate against a cancer cell comprising an antigen that specifically interacts with the tumor-targeting small molecule.

4. The method of claim 1, wherein the tumor-targeting small molecule is selected from the group consisting of:
Folic acid, or derivatives thereof,
Biotin,
EGF,
Integrin binding proteins,
RGD peptides and their derivatives,
extracellular matrix-homing peptides,
tumor associated macrophages-targeting agents,
EGFR targeting peptide,
Angiopep-2,
peptides targeting aberrant cellular signaling pathways,
PSMA binders, and
FAP binders.

5. The method of claim 1, wherein the tumor-targeting small molecule is folic acid, or a derivative thereof, and wherein the antigen that specifically interacts with the tumor-targeting small molecule is folate receptor alpha (FRα).

6. (canceled)

7. The method of claim 1, wherein the ADC has the formula A-L, wherein A is an antibody, or an antibody fragment, and wherein L is a linker comprising the cytotoxic molecule and the tumor-targeting small molecule.

8. The method of claim 7, wherein the linker is conjugated to the side chain of a glutamine residue of the antibody.

9. The method of claim 8, wherein the glutamine residue to which the linker is conjugated (i) is comprised in an Fc domain of the antibody; or (ii) has been introduced into the heavy or light chain of the antibody by molecular engineering; or wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

10. (canceled)

11. (canceled)

12. The method of claim 8, wherein the linker is conjugated to the side chain of the glutamine residue of the antibody via a lysine residue, a cadaverine moiety or a PEG amine moiety.

13. The method of claim 12, wherein the lysine residue is comprised in a peptide linker.

14. (canceled)

15. The method of claim 13, wherein the net charge of the peptide linker is neutral or positive; and/or wherein the peptide linker comprises no negatively-charged amino acid residues; and/or wherein the peptide linker comprises at least one of an arginine or histidine residue.

16. (canceled)

17. (canceled)

18. The method of claim 13, wherein the peptide linker comprises the sequence motif arginine-lysine (RK) or histidine-lysine (HK).

19. The method of claim 13, wherein the peptide linker comprises the sequence RKAA, RKA, ARK or RK-Val-Cit.

20. The method of claim 13, wherein the tumor-targeting small molecule and/or the cytotoxic molecule are linked to the N- or C-terminus of the peptide linker or to a side-chain of an amino acid residue comprised in the peptide linker.

21. The method of claim 13, wherein the tumor-targeting small molecule is linked to the N-terminus of the peptide linker and wherein the cytotoxic molecule is linked to the C-terminus of the peptide linker, or vice versa.

22. The method of claim 21, wherein the tumor-targeting small molecule is linked to the N- or C-terminus of the peptide linker via one or more PEG moieties.

23. The method of claim 7, wherein the linker is conjugated to the side chain of a cysteine residue of the antibody.

24. The method of claim 7, wherein the cytotoxic molecule is linked to the linker or peptide linker via a self-immolative moiety.

25. The method of claim 1, wherein the cytotoxic molecule is at least one selected from the group consisting of
a pyrrolobenzodiazepine;
an auristatin;
a maytansinoid;
a duocarmycin;
a nicotinamide phosphoribosyltransferase (NAMPT) inhibitor;
a tubulysin;
an enediyne;
an anthracycline derivative (PNU);
a pyrrole-based kinesin spindle protein (KSP) inhibitor;
a cryptophycin;
a drug efflux pump inhibitor;
a sandramycin;
an amanitin; and
a camptothecin.

26. The method of claim 1, wherein the antibody is an IgG antibody; and/or wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, and/or
wherein the antibody is selected from the group consisting of: Brentuximab, Trastuzumab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab and Enfortumab.

27. (canceled)

28. (canceled)


SEQUENCE LISTING (4 sequences)

SEQ ID NO: 1 - protein, 447 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSVSASVGDRVTITCRASQGISGWLAWYQQKPGKAPKFLIYAASTLNSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 213 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC